(19) United States
(12) Patent Application Publication
(10) Pub. No.: US 2019/0029879 A1
(43) Pub. Date: Jan. 31, 2019

(54) HEAT PADS COMPRISING SPIRAL HEAT CELLS

(71) Applicant: BEIERSDORF AG, Hamburg (DE)

(72) Inventors: Karl-Heinz WOELLER, Hamburg (DE); Pia RUECKER, Hamburg (DE); Jens NIERLE, Hamburg (DE)

(73) Assignee: BEIERSDORF AG, Hamburg (DE)

(21) Appl. No.: 15/741,318

(22) PCT Filed: Jun. 28, 2016
(86) PCT No.: PCT/EP2016/064937

§ 371 Date: Apr. 27, 2018

(30) Foreign Application Priority Data
Jul. 3, 2015 (DE) 10 2015 212 494.0

(51) Int. Cl. A61F 7/03

(52) U.S. Cl. CPC A61F 7/034; A61F 2007/0226

(57) ABSTRACT

The invention relates to heat products for single treatment and/or as self-therapy in the event of acute, recurrent and/or chronic states of pain, comprising heat-generating materials arranged in spiral form.

The invention relates to heat products for single treatment and/or as self-therapy in the event of acute, recurrent and/or chronic states of pain, comprising heat-generating materials arranged in spiral form.

Heat products for single treatment and/or as self-therapy in the event of acute, recurrent and/or chronic states of pain in muscles and/or joints, in the event of feelings of stiffness, nerve pain, rheumatism, menstrual pain etc. have been enjoying increasing popularity with users since the 1990s in Europe, North America and Australia and have been generating accompanying constant growth in sales for manufacturers. One example to be mentioned here is the HANSAPLAST heat therapy pad.

The origin of heat pads with the generation of heat by exothermic reaction lies in Asia. To date, the only globally acknowledged standard for the definition of test parameters and test methods for self-heating products is the Japanese Industrial Standard (JIS) S 4100 “Disposable body warmers” from 1996, even though the demands from the market and customers have made the minimum demands of this standard clearly out of date with regard to duration of use.

Heat-generating material mixtures have long been known in the prior art; reference is made here by way of example to WO2013054138.

The technological basis of these heat products is generally the controlled and exothermic oxidation of iron powder or finely divided iron filings present in a material mixture, wherein the material mixture includes at least carbon powder, water and a salt as electrolyte or ion former. Further constituents that may be present in the heat-generating material mixture include, for example, charcoal chips, humectants, agglomeration assistants, binders, metal salts, organic or inorganic fillers and many other substances, in order to establish the desired end product properties.

These material mixtures that are familiar to the person skilled in the art for release of heat through controlled exothermic oxidation may be used in a heat pad in the form of powder or in compacted form as granules, agglomerates, beads, pellets or tablets. For use in a heat pad, the heat-generating material mixtures described are typically incorporated in segmented form in what are called “heat cells”.

These heat cells typically form by virtue of a defined amount of heat-generating material mixture being fully surrounded by two polymer films bonded to one another, in which case at least one polymer film must be oxygen-permeable by definition.

The heat cells may also take the form of a closed tube or a hose or a pouch. Heat-generating material mixtures may also be applied to thermoforming films and then be sealed in (e.g. EP 1782787).

In the heat pad, these heat cells can then be fixed in different sizes, different numbers and different configurations between two carrier substrates bonded in the manner of a laminate, for example by adhesive bonding or fusion, in which case the carrier material adjoining the oxygen-permeable polymer film of the heat cell must also be oxygen-permeable. These carrier substrates may be films, wovens, nonwovens, gauze or any other carrier substrate suitable for application to the human body.

In one form of application, the heat pads are modified so as to be self-adhesive on the side that will later face the skin. For this purpose, an appropriate skin-friendly adhesive is applied to the carrier substrate. The adhesive can be applied over all or part of the area, for example in the form of a wave, in dots, or in the form of a continuous or interrupted grid with different grid sizes and shapes. Adhesives known include a multitude of different adhesives from the prior art, for example acrylate adhesives, synthetic rubber adhesives or silicone adhesives.

In order to avoid direct contact of the skin with the adhesive of the heat pad, for example in the case of users particularly sensitive to adhesives, the heat pads can also be bonded to the inside of near-skin items of clothing such as T-shirts or vests. However, the heat-insulating air cushion which is necessarily present between the skin and heat pad reduces good heat transfer from the heat pump to the body.

Another known method is not to modify the heat pads so as to be self-adhesive, but instead to secure them to the body in prefabricated pockets or recesses of belts, tubular bandages, bodices, vests or similar fixing aids that have been specially manufactured for this purpose. The fixing of heat pads by means of plasters or medical adhesive tapes is also known.

In the case of use of heat pads as heat belts, the corresponding heat cells are fixed in the manner of a laminate between a skin-remote and a skin-facing carrier substrate. Since the heat cells in the heat pad or heat belt are activated by contact with atmospheric oxygen, the heat pads, directly after production, have to be sealed into air-impermeable packaging. It is only before use that the heat pad is then removed from the packaging and contact with oxygen initiates the exothermic process (oxidation of the iron present in the heat-generating material mixture).

According to JIS S 4100, the generation of heat should have advanced after 30 min to such an extent that a use temperature on the skin between 40 and 45° C. is attained. A disadvantage in the case of this type of heat pads is the fact that the material mixture which is pulverulent prior to use blocks together (forms lumps, sinters together) to form a stiff solid body over the entire surface during the heat generation process.

The heat cells are thus no longer capable, in the event of user movement, of following the changing body surface contours; it is even possible that particular movements are inhibited or hindered as a result. But even in the case of less hindered movements, these rigid heat cells are perceived as troublesome by many users.

Moreover, such stiff heat cells can mean that the heat pads no longer have complete contact with the skin surface in the course of and/or after user movements and hence the full healing-active heating power of the product is not transmitted to the user.

One means of reducing the adverse stiffening of heat pads resulting from the ‘hardening’ heat cells is disclosed, for example, in DE 69729585 T2. The person skilled in the art knows from this that, when the heat cells which have typically been large-area rectangles to date are replaced by small-area round, oval or rectangular heat cells with a specific geometric arrangement relative to one another, heat cell-free axes occur in multiple directions across the whole area of the heat pad, which can act as joint axes when the user moves.

However, a disadvantage of this solution is that these smaller heat cells also block to become individually stiff single cells.

However, the joint axes have the disadvantage that the region of the heat cells of the heat pad has to be supported by stiff or semirigid materials in order to overcome deformations or folding during application.

There are particular embodiments of heat pads for particular application cases. A popular form is that of heat belts for use in the lumbar region, as disclosed, for example, in US1996/0777830.

It was thus an object of the invention to remedy the disadvantages of the prior art, especially to provide improved and more elastic heat pads.

Heat products according to JIS S 4100 in which the base area containing heat cells is not fully bonded to the skin to be treated, for example products bonded only at 2 to 3 points analogously to ThermaCare® heat pads or S-O-S® heat wraps, have the disadvantage that the non-tacky region of the heat cells which is generally mounted in the middle rises up from the skin when the user moves, resulting in a heat-insulating air layer between the product and the skin. Since air is a very good heat insulator, this results in a reduction in the healing effect of the heat products.

In order to prolong the release of heat or to achieve more uniform release of heat, the prior art discloses (for example in US 2012/0150268 and JP 2005/137465), adding phase change materials (PCMs) to the heat-generating material mixtures.

PCMs are substances that can store or release large amounts of energy when they melt or solidify at a particular temperature. This means that PCMs are a latent heat store and a suitable material for significantly reducing the effects of possible local overheating of heat cells for use on humans. When PCMs, in the course of heating, reach the temperature at which they change phase, for example the melting temperature, the absorb large amounts of heat energy at virtually constant temperature until all the material has melted. When the ambient temperature drops again, the PCMs solidify and release the energy stored again.

Materials suitable as PCMs for use in heat cells are, for example, alkanes having 14 to 30 carbon atoms or mixtures of these alkanes. Corresponding materials and the use thereof are disclosed, for example, in US 2012/0150268.

A disadvantage of the PCMs described is that, as they melt, they combine with the rest of the components of the heat-generating material mixture in a heat cell and hence likewise block to give a stiff overall structure.

The use of microencapsulated PCMs for heating and cooling pads is also known to the person skilled in the art from the prior art (for example US 2003/0109910 and CA 2289971). However, in the case of the known solutions, the microencapsulated PCM is embedded in each case into solutions or gels that have to be heated in a microwave or a water bath before use. There are no known heat-generating material mixtures having a proportion of microencapsulated PCMs that are suitable for heat cells.

It was surprising and unforeseeable to the person skilled in the art that the stiffness of “through-oxidized” fuel cells can be distinctly reduced and wear comfort to the user can be distinctly enhanced when the heat cells are configured not as flat individual structures such as rectangles, circles, ellipsoids, rhomboids etc. but as fine-limbed spirals.

The invention therefore provides heat pads having one or more heat cells, characterized in that the heat cells have a spiral shape.

Nor was it foreseeable to the person skilled in the art that the use of microencapsulated PCMs can achieve a reduction in stiffness since they do not combine with the rest of the components of the heat-generating material mixture and hence increase the mobility of the cell composite.

It is also within the scope of the invention that the heat pads of the invention have one or more heat cells containing heat-generating substance mixtures comprising microencapsulated PCMs.

Encapsulated PCMs are commercially available, for example, under the Lurapret® or Micronal® trade name from BASF.

Heat pads of the invention may have a self-adhesive coating for direct fixing in the area of the body to be treated or may be arranged over the area of the body to treated by means of additional fixing aids.

Spirals have the potential to absorb force via tension owing to their curved geometry. In the case of an Archimedean spiral, at maximum, until the curved extent between two opposing points where forces act in opposite directions becomes a straight line or a brittle material breaks beforehand. The absolute length of the spiral arm affected by a force, and hence indirectly the size of a spiral, thus determines the decrease in the stiffness of a heat cell designed in such a way.

By contrast with flat heat cells in which a minimum size and hence a multitude of separately arranged heat cells is advantageous for a low stiffness of the overall product, there is a decrease in the stiffness of the end products in the case of spiral heat cells with the size of the heat cells. The size of the heat cells can be determined via the circumference and also via the number of turns of the individual spiral arms.

The spiral heat cell may be formed in terms of its basic shape, for example, as an Archimedean, logarithmic, hyperbolic or Fermat spiral, lituus spiral, root spiral, triskelion or clothoid, or of one of or a plurality of any desired combination forms of all known spirals.

More particularly, it is advantageous when a heat cell of the invention takes the form not of an Archimedean spiral (FIG. 1) but of a Fermat spiral (FIG. 2).

The two-arm Fermat spirals, by virtue of the through-connection of the spiral arms over the entire spiral area, offer the maximum potential to reduce the stiffness of heat cells. A further positive aspect of Fermat spirals is that both spiral arms, proceeding from the middle, end at the outer edge of the overall structure and therefore can be joined in turn to the outer ends of other spirals, especially preferably Fermat spirals, in order to further reduce the stiffness of the overall structure.

Multi-arm Fermat spirals have somewhat lower potential for prevention of stiffness of heat cells, but do offer the option of joining to multiple spirals, again especially Fermat spirals, and hence a further reduction in stiffness.

Particular preference is given to the form of one or more triskelions or triple spirals (FIG. 3), in order to form a network of coherent spirals.

A further great advantage, which is novel for the execution of heat cells as spirals, is the fact that spirals have potential for mobility not just in the x and y axis but additionally also in the z axis at right angles to the plane of the spiral. This is of particular relevance to users of correspondingly elaborated heat products when the products are to be employed over parts that protrude from the body in movement, for example over joints or the shoulder blade.

A self-adhesive or non-self-adhesive heat product according to the invention analogous to JIS S 4100 may be a spiral heat cell or any plurality of individual (FIG. 5) or mutually bonded (FIG. 4) spiral heat cells. The spiral heat cells need not be exactly circular, but may also be oval, ellipsoidal, square or elongatedly rectangular. Especially for large-area heat products for use in the lumbar region of the back, elongated rectangular spiral forms are advantageous. To obtain an elongated rectangular spiral form with a uniform distance of the spiral arms from one another, the spiral arms in the x axis with fluid transitions may be distinctly broader than in each case on the y axis.

The base area of a heat cell of the invention in spiral form may be from 0.75 cm² to 1300 cm², preferably 3 cm² to 620 cm², more preferably 20 cm² to 320 cm², most preferably 50 cm² to 250 cm², meaning only the area covered by the heat-generating material mixture.

The width of a spiral arm may be from 0.1 cm to 5 cm, preferably 0.2 cm to 3 cm, more preferably 0.25 cm to 2 cm. The widths of spiral arms in the x and y axis of a spiral heat cell projected onto a flat surface may likewise be different, and possibly also alternating. This is a good compensation means for a homogeneous user product especially in the case of large-area, elongated rectangular spirals as heat cells. FIG. 4 shows a heat cell formed from four coherent spirals, where the arms of the two outer spirals of the chain have a smaller width than the arms of the two inner spirals. The heat output of the inner spirals is increased compared to the outer spirals as a result.

A particular advantage of the spiral execution of the heat cells is considered to be that comparatively narrow spiral arms, by contrast with heat cells that are flat overall, have a much lower fracture resistance. If a force is exerted on the heat cell on application through user movement, the spiral arms will break much more easily in parallel with the direction of movement than flat heat cells owing to the lower width of the material composite of the oxidized mixture. As a result, a joint line matched specifically to the user can form in the heat cell in each case exactly in parallel with the respective maximum movement stress.

It is not possible to give an average distance of the spiral arms from one another owing to the underlying geometries, particularly in the case of logarithmic and hyperbolic spiral arms and especially owing to the common origin of the spiral arms in Fermat spirals. Preferred distances between the turns of respective individual spiral arms, within the overall area of a heat spiral, are 0.1 cm to 5 cm, more preferably 0.2 cm to 4 cm and most preferably 0.2 cm to 3 cm.

In all geometric embodiments of spiral heat cells, the distances in the configurations should be chosen such that the heat radiated from individual spiral arms overlaps very substantially in the user's skin in order to give a homogeneous feeling of warmth over the entire treatment area.

With regard to their heights and thicknesses, spiral heat cells of the invention can differ distinctly over the spiral area. For instance, the thickness of a spiral, toward the midpoint thereof, can distinctly increase or else decrease compared to the outer regions, according to the desired user properties.

The height of the spiral area or of the spiral arms of a heat cell may vary from 0.05 cm to 1.5 cm, preferably 0.05 cm to 1.0 cm, more preferably 0.05 cm to 0.7 cm, most preferably 0.1 cm to 0.5 cm.

The degree of filling of a spiral heat cell with heat-generating material mixture is preferably 50% to 100% of the maximum fill volume, more preferably 70% to 100%, most preferably 90% to 100%.

The weight of a spiral heat cell of the invention is preferably 1 g to 400 g, preferably 2 g to 300 g, more preferably 4 g to 250 g, most preferably 4.5 g to 220 g.

The heat pads of the invention may comprise one or more spiral heat cells. Preferably, the heat pads of the invention include multiple spiral heat cells, where the heat cells may also be joined or interwoven.

Wholly or partly self-adhesive heat pads are preferably elongated rectangular with ends tapering to a cone. The dimensions of the heat pad are highly dependent upon the site of use. Heat pads intended for the heat treatment of the back, for example, may be up to 40 cm long and 20 cm wide. Universally usable heat pads preferably have a length between 20 cm and 30 cm and a width between 10 cm and 15 cm.

It may be advantageous to supply heat pads in various sizes, matched to different body sizes, for the same site of application.

More preferably, these heat pads contain four Fermat cells arranged in succession, most preferably joined to one another, in identical or different configuration in terms of size, shape, length, width, weight, and length and distance of the spiral arms from one another. (FIG. 6)

In a further preferred execution, heat pads of this kind are manufactured in the form of a triangle having concave recesses in the sides and rounded off at the corners. FIG. 7 shows, by way of example, a “triangular” heat pad with four separate heat cells, wherein there is a triskelion-shaped heat cell at the center surrounded by three heat cells in the geometry of Fermat spirals.

Preferably, heat pads of the invention contain, centrally, in the middle, a heat cell with the geometry of a three-arm Fermat spiral connected at the end point of each spiral arm to a further heat cell centered in the direction of the tips of the triangle in the form of a Fermat spiral (FIG. 8).

Heat pads of the invention in the form of a heat belt contain preferably 1 to 8, more preferably 2 to 4, spiral heat cells optionally partly joined to one another via the spiral arms. The heat cells are preferably distributed over a heat-releasing base area of less than 25 cm by 35 cm and may be arranged either one alongside another or one on top of another.

In a further form of a reusable heat belt, one or more non-tacky heat pads are positioned in appropriately prefabricated pockets.

The heat belt has belt elements on either side, which may consist of a multitude of materials and forms and different elasticity known to those skilled in the art, and end in a common fastener system on the longitudinal axes. Preferably, the common fastener system consists of parts of hook and loop fasteners, adhesive fasteners, hook and eye fasteners or (press-)studs mounted horizontally or vertically with respect to one another.

The material of the pockets for accommodating the heat pads must be sufficiently extensible and elastic to assure reliable holding of the heat pads.

In a heat pad of the invention, spiral heat cells may preferably be incorporated in the manner of a laminate between longitudinally elastic, more preferably bielastic, carrier materials. In the case of incorporation between merely longitudinally elastic carrier materials, for a maximum reduction in the stiffness of the end product, it should be ensured that the spiral heat cell(s) is/are arranged with their maximum diameter in the direction of the elasticity of the carrier materials.

Suitable longitudinally elastic or bielastic carrier materials are commercially available in various forms. Longitudinally elastic, for example, in the form of elastic fabric from Kümpers, Rheine, Germany, a fabric composed of cotton containing 4% Lycra, with a basis weight of around 180 g/m² and an extensibility of more than 200% of its starting length. Or, for example, Article 016 from KOB, Wolfstein, Germany, a fabric consisting of 70% viscose and 30% polyamide with an extensibility of 60%.

Bielastic fabrics are likewise obtainable from KOB, for example article 023 composed of 100% cotton with a longitudinal extensibility of 85% and transverse extensibility of 40%, or article 053 composed of a 100% PET fabric with a longitudinal extensibility of 25-40% and a transverse extensibility of >40%.

Further bielastic materials of good suitability are also available, for example, from Innovatec, Troisdorf, Germany, for example thermoplastic polyurethanes (TPU) having a basis weight of 75 g/m² and a longitudinal extensibility of 300% and a transverse extensibility of 330%.

Suitable carrier materials for heat cells of the invention may also advantageously be modified in a heat-insulating manner on the side remote from the skin. This at least partial heat insulation reduces release of heat into the surrounding space; in this way, it is possible to save on heat-generating materials in the cells and reduce the total weight of the end product. The heat insulation of the carriers can be generated in various ways, for example by metal foil coatings or else by incorporation of natural residues from coffee husk processing. Products using the latter technology are commercially available, for example, under the NILIT® Heat name.

Naturally, every user of heat pads has their own subjective perception of the amount of heat released in each case. The temperature range specified in JIS S 4100 for these products may be perceived by the different user as just right, or else, with a multitude of nuances in between, as too cold or too hot. One means of providing a remedy here for the individual user is to provide the skin-remote side of the heat cell composite of products of the invention with only lightly adhering materials of different oxygen permeability, preferably multiple materials with reducing oxygen permeability from the inside outward in laminate form. If the heat output is insufficient for the user of such a product, they can remove the outer material layer and more oxygen can reach the exothermic process within a heat cell. This increases the reaction rate and hence the resulting temperature, but with a corresponding reduction in the possible total utilization time of the product. By removal of further material layers, this process can correspondingly be influenced further in an individual manner by the user.

The principle described is also applicable in the reverse manner, in that adhering material layers of limited oxygen permeability are correspondingly added to the heat product, which are mounted additionally on the outer side of the heat product by the user according to their personal comfort temperature in order to reduce the oxygen supply, which in turn leads to a reduced overall temperature and to a prolonged duration of use.

Both the principles outlined above are also executable with materials of identical oxygen permeability; in that case, merely additive and subtractive mechanisms of control in the course of the reaction are manifested here.

A further advantageous means of better utilization or release of the heat energy from cells of the invention consists in the addition of phase changing materials (PCMs) to the carrier materials, but more preferably directly as a component of the exothermic material mixture.

For use in exothermic heat mixtures in the spiral form of the invention, particular preference is given to encapsulated PCMs having a capsule diameter which is less than the thickness of the spiral and is advantageously 0.5 to 1.5 mm. Since the capsules retain their outer shape during the temperature transitions of the PCMs, they do not enter into any bond with the reacting and slowly through-hardening heat mixture and, because of their size, can thus serve as inherent joints or intended fracture sites in the heat spirals under expenditure of force by the user and hence ensure a distinct reduction in stiffness when employed on the body.

The disadvantage of the poor heat transfer in heat pads that are not bonded over the full area can be reduced by applying a lenticular, semi-convex layer of heat-conducting polymers on the skin-facing side of the heat pad in the region of the heat cells. This lenticular layer, in terms of areal extent, may be either circular or else ellipsoidal or oval or irregular.

Preference is given here to polymer layers of silicone, since silicones have very good elasticity and heat conduction properties (for example silicone resins at RT from 0.15 to 0.32 W/mK), but especially also comparatively good compression characteristics from 15% to 30%. Lenticular silicone layers having a thickness of 0.01 cm to 2.5 cm and a diameter of not more than 2 cm to 20 cm are therefore of good suitability for assuring full skin contact of the exothermic heat cells for optimal heat transfer even in the event of user movement. More preferably, this layer is manufactured from silicone polymers having a Shore A hardness of not more than 50.

Heat pads of the invention using spiral heat cells may be provided with heat displays. Since the subjective perception of heat by the user can generally decrease with increasing application time as a result of habituation effects and the heat product, as a result, can already be removed before the end of the indicated treatment time by the user, a visual temperature check is advantageous. Corresponding indicators that can visualize the temperature via chemical color reactions are known to those skilled in the art, for example from US 2009/0149925.

In the case of heat belts for multiple use, in which the heat pads containing heat cells are to be renewed before every use, it may be advantageous to incorporate the heat indicators into the belt, which is not to be renewed.

It is advantageous and within the scope of the invention to equip the heat pads of the invention with active ingredients for assistance of therapy. These active ingredients may be stored on the skin-facing side of the products, incorporated in a corresponding depot, until use, or else, in the case of heat pads that have been modified to be self-adhesive, incorporated into the adhesive matrix (called monolithic systems).

Heat pads of the invention may, for example, also be modified with active hyperemizing ingredients, for instance antiphlogistics and/or analgesics, such as natural active ingredients of cayenne pepper or synthetic active ingredients such as nonivamide, nicotinic acid derivatives, preferably benzoyl nicotinate or propyl nicotinate. Advantageous active ingredients are capsaicin (N-(4-hydroxy-3-methoxybenzoyl)-8-methyl-trans-6-nonenamide), nonivamide, benzoyl nicotinate or benzoyl nicotinate.

Non-steroidal antirheumatics are likewise suitable as active ingredients, for example glycol salicylate, flufenamic acid, ibuprofen, etofenamate, ketoprofen, piroxicam, indomethacin. Likewise of good suitability are antiphlogistics such as acetylsalicylic acid, antipruritics, for example polidocanol, isoprenaline, crotamiton, or local anesthetics, for example lidocaine, benzocaine.

It is advantageous to dope heat pads of the invention with active ingredients that have a positive effect on the condition of the skin. These active ingredients do not just lead to better skin compatibility of the self-adhesive heat pads but also actively improve the outward appearance of the skin, for example in the case of wrinkles, scars or cellulite. Particularly preferred active ingredients here include bioquinones, especially ubiquinone 6210, creatine, creatinine, carnitine, acetylcarnitine, biotin, isoflavone and isoflavonoids, genistein, arctiin, cardiolipin, liponic acid, anti-freezing proteins, hops extracts and hops malt extracts, and/or substances that promote the restructuring of the binding tissue, and likewise isoflavonoids and isoflavonoid-containing plant extracts, for example soya extracts and clover extracts. It is also possible active ingredients to assist skin functions in the case of dry skin, for example vitamin C, biotin, carnitine, creatine, creatinine, propionic acid, glycerol, green tea extracts and urea.

Active ingredients used for supporting aromatherapy may additionally be essential oils, for example in the case of use of heat pads of the invention for menstrual complaints. The essential oils here may not just be incorporated into the skin-facing carrier substrate but especially also into the skin-remote carrier substrate. More preferably, the active ingredients are in encapsulated form.

Essential oils are understood to mean concentrates obtained from plants that are used as natural raw materials, mainly in the perfume and food industry, and consist to a greater or lesser degree of volatile compounds. Examples of these compounds include 1,8-cineol, limonene, menthol, borneol and camphor. The term “essential oils” is often used for the volatile ingredients that are still present in the plants. In the actual sense, however, essential oils are understood to be mixtures of volatile components that have been produced from plant raw materials by steam distillation.

Essential oils consist exclusively of volatile components having melting points generally between 150 and 300° C. They comprise predominantly hydrocarbons or monofunctional compounds such as aldehydes, alcohols, esters, ethers and ketones. Parent compounds are mono- and sesquiterpenes, phenolpropane derivatives and longer-chain aliphatic compounds.

In some essential oils, there is one dominant ingredient, for example eugenol in clove oil at more than 85%, but other essential oils are mixtures of complex compositions of the individual constituents. The organoleptic properties are often shaped not by the main components but by secondary or trace constituents, for example by the 1,3,5-undecatrienes and pyrazines in galbanum oil. In many of the essential oils of commercial significance, the number of components identified goes into the hundreds. Very many ingredients are chiral, and it is very often the case that one enantiomer is predominant or present exclusively, for example (−)-menthol in peppermint oil or (−)-linalyl acetate in lavender oil.

Preferred essential oils include oleum eucalypti, oleum menthae piperitae, oleum camphoratum, oleum rosmarini, oleum thymi, oleum pini sibricum and oleum pini silverstris, and the terpenes 1,8-cineol and levomethanol.

### FIGURES

FIG. 1 shows an Archimedean spiral. It forms when, in a rotary motion, the radius grows proportionally with the angle of rotation.

FIG. 2 shows a Fermat spiral.

FIG. 3 shows a triskelion.

FIG. 4 shows, by way of example, a heat pad 1 having one heat cell 2, wherein the heat cell has the shape of four Fermat spirals 2′, 2″, 2′″ and 2″″ joined to one another.

FIG. 5 shows, by way of example, a heat pad 3 having two heat cells 4, 4′, wherein the heat cells each have the shape of Fermat spirals.

FIG. 6 shows, by way of example, an elongated rectangular heat pad 5 with ends tapering to a cone, having a heat cell 6, wherein the heat cell has the shape of four Fermat spirals joined to one another.

FIG. 7 shows, by way of example, a ‘triangular’ heat pad 7 having four separate heat cells 8, 9, 10, 11, wherein there is a triskelion-shaped heat cell 8 at the center surrounded by three heat cells 9, 10, 11 in the geometry of Fermat spirals.

FIG. 8 shows, by way of example, a ‘triangular’ heat pad 12 having one heat cell 13, wherein the heat cell has at the center a geometry of a three-arm Fermat spiral 13′ connected at the end point of each spiral arm to a further heat cell section 13″, 13′″, 13″″ centered in the direction of the tips of the triangle in the form of a Fermat spiral.

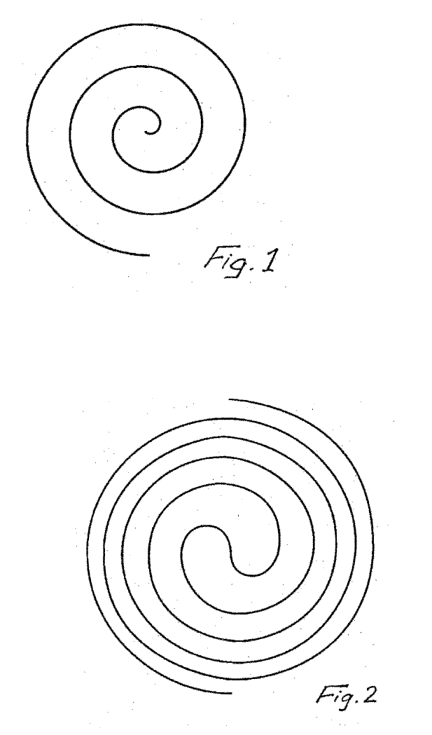

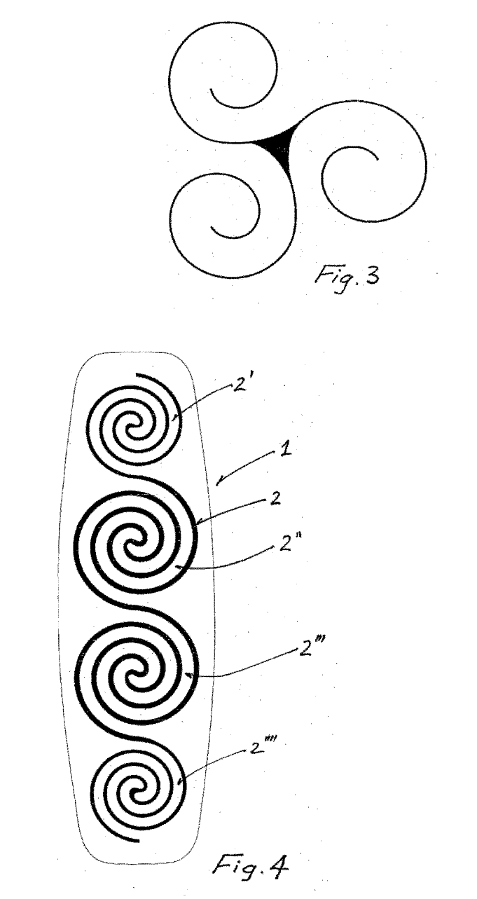

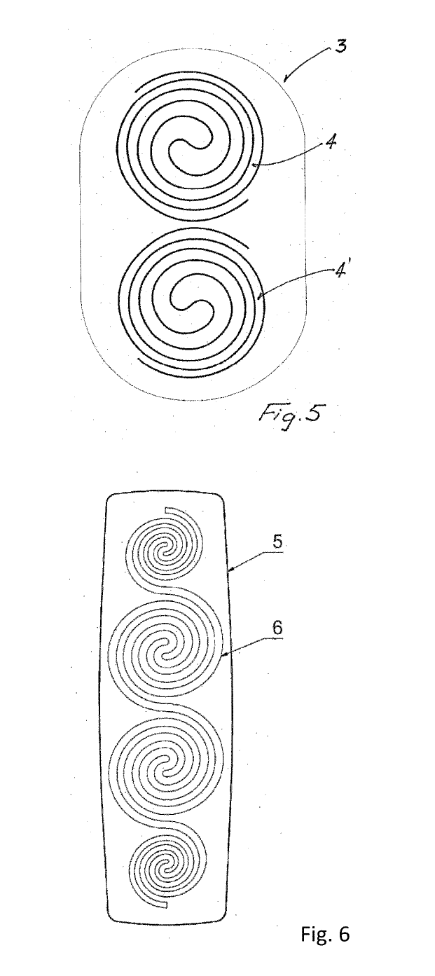

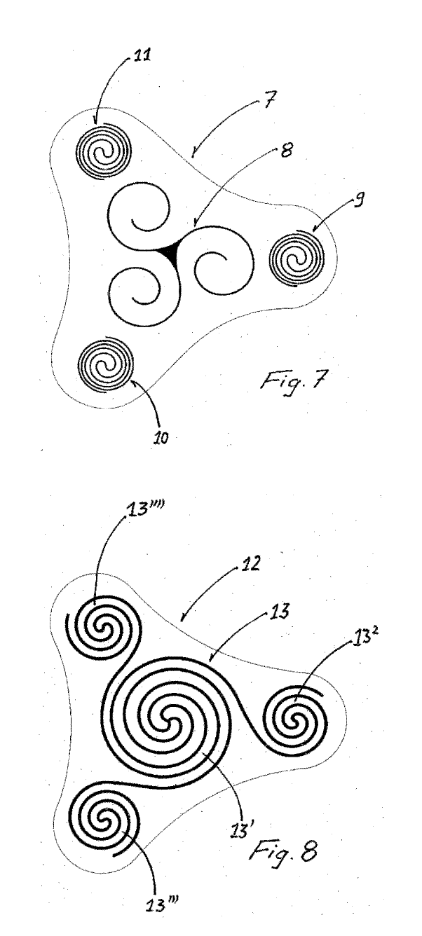

1.-14. (canceled)

15. A heat pad comprising at least one or more heat cells, wherein at least one of the one or more heat cells has a spiral shape and comprise a heat-generating substance mixture which comprises a heat-generating material mixture comprising at least iron powder and carbon powder.

16. The heat pad of claim 15, wherein at least one of the one or more heat cells is shaped as an Archimedean spiral.

17. The heat pad of claim 15, wherein at least one of the one or more heat cells is shaped as a Fermat spiral.

18. The heat pad of claim 15, wherein at least one of the one or more heat cells is shaped as a triskelion spiral.

19. The heat pad of claim 15, wherein at least one of the one or more heat cells is shaped as a multiple spiral.

20. The heat pad of claim 15, wherein the spiral has an oval or angular shape.

21. The heat pad of claim 15, wherein at least a part of a surface of the pad has an adhesive layer thereon.

22. The heat pad of claim 15, wherein a defined amount of the heat-generating material mixture is fully surrounded by two polymer films bonded to one another, at least one of the polymer films being oxygen-permeable.

23. The heat pad of claim 15, wherein a defined amount of heat-generating material mixture is enclosed in a tube or a hose, the tube or hose consisting of an oxygen-permeable material.

24. The heat pad of claim 15, wherein the one or more heat cells are exchangeable.

25. The heat pad of claim 15, wherein the heat pad comprises at least four heat cells in spiral form.

26. The heat pad of claim 25, wherein the heat pad is in the form of a triangle which has concave recesses in its sides and is rounded off at its corners.

27. The heat pad of claim 25, wherein the heat pad has a rectangular elongated form.

28. The heat pad of claim 27, wherein the heat pad comprises at least two cells in spiral form arranged successively in longitudinal direction.

29. The heat pad of claim 26, wherein the heat cells in spiral form are configured as Fermat spirals bonded to one another.

30. The heat pad of claim 28, wherein the heat cells in spiral form are configured as Fermat spirals bonded to one another.

31. The heat pad of claim 15, wherein the heat-generating material mixture comprises, in at least one heat cell, a microencapsulated phase change material (PCM).

32. The heat pad of claim 15, wherein a lenticular, semi-convex layer of heat-conducting polymers is present on a skin-facing side of the heat pad in a region of the heat cells.

33. A heat belt, wherein the heat belt comprises at least one heat pad according to claim 15.

34. The heat belt of claim 33, wherein the heat belt is configured to enable joining in the form of a loop, enwrapping a body, by belt elements mounted on a longitudinal axis and ending in a fastener system.
